(19) United States
(12) Patent Application Publication
(10) Pub. No.: US 2025/0012803 A1
(43) Pub. Date: Jan. 9, 2025

(54) METHODS FOR ASSESSING AN IMMUNE RESPONSE AND PREDICTING CANCER RECURRENCE AND ASSESSMENT OF CURRENT AND PRIOR CANCER STATUS

(71) Applicant: Greenwich LifeSciences, Inc., Stafford, TX (US)

(72) Inventors: Snehal S. Patel, Sugar Land, TX (US); Jaye Thompson, Woodlands, TX (US)

(21) Appl. No.: 18/709,972

(22) PCT Filed: Nov. 18, 2022
(86) PCT No.: PCT/US2022/050474

§ 371 Date: May 14, 2024

Related U.S. Application Data
(60) Provisional application No. 63/280,958, filed on Nov. 18, 2021.

(51) Int. Cl. G01N 33/574

(52) U.S. Cl. CPC G01N 33/57415; G01N 2333/82

(57) ABSTRACT

The present disclosure provides methods for using an immune response to a peptide, such as a peptide that correlates with a cancer, for instance an oncogenic protein, to determine if a subject is at an increased risk for recurrence of a cancer including increased risk of reduced time to recurrence of a cancer, if a cancer is present in the subject, or if the subject had a cancer prior to the testing and developed an immune response to the peptide. The methods include testing a subject's immune response to a peptide, such as a peptide that correlates with a cancer, for instance an oncogenic protein. The testing can occur at baseline, after baseline, or before baseline. In one embodiment, the peptide is a peptide of the oncogenic protein HER2/neu, such as GP2.

### CROSS-REFERENCE TO RELATED APPLICATIONS

This application claims the benefit of U.S. Provisional Application Ser. No. 63/280,958, filed Nov. 18, 2021, which is incorporated by reference herein in its entirety.

### SEQUENCE LISTING

This application contains a Sequence Listing electronically submitted via EFS-Web to the United States Patent and Trademark Office as an ASCII text file entitled “0629.000002WO01.xml” having a size of 2 kilobytes and created on Nov. 18, 2022.

The information contained in the Sequence Listing is incorporated by reference herein.

### BACKGROUND

HER2/neu (human epidermal growth factor receptor 2, also referred to herein as HER2) protein is a cell surface receptor protein that is expressed in a variety of common cancers, including 75% of breast cancers. In breast cancers the protein can be expressed at low (1+), intermediate (2+), and high (3+ or over-expressor) levels. Resection of the tumor by surgery is helpful in treatment, and further therapy is typically recommended. There are a number of therapies approved for HER2/neu-expressing breast cancers. For instance, a HER2/neu-expressing subject can receive trastuzumab (Herceptin®) or a trastuzumab-based derivative before surgery or in the first year after surgery as a follow up therapy to reduce the likelihood of recurrence. However, some patients do not respond to the trastuzumab-based therapy and recur within five years of the surgery or thereafter.

### SUMMARY OF THE APPLICATION

GP2 is a 9 amino acid transmembrane peptide of the HER2/neu protein. GP2, in combination with granulocyte-macrophage colony-stimulating factor (GM-CSF) which combined are called GLSI-100, is a candidate therapy to reduce cancer recurrence in breast cancer patients with tumors expressing any degree of HER2/neu. It was found that a subject having a baseline delayed type hypersensitivity (DTH) immune response to GP2, prior to any treatment with GP2, has an increased risk of experiencing a breast cancer recurrence compared to those not having a baseline DTH immune response to GP2 prior to any treatment with GP2. It was also found that administration of GLSI-100 safely elicited a potent immune response at the site of injection. The injection site reactions (ISRs) resulting from GLSI-100 administration correlate to and can be used to complement or replace a baseline DTH immune response to GP2. Thus, as described herein an immune response such as, but not limited to, a positive DTH immune response to a peptide at baseline, an injection site reaction, or a combination thereof, can be indicative of whether the subject is more likely to have a recurrence of a cancer. The peptide can be one that is a peptide of a protein correlated with a cancer, such as a peptide of an oncogenic protein. In another embodiment, a positive DTH immune response to a peptide at baseline, an injection site reaction, or a combination thereof, can also be used to predict whether (i) a cancer is present in the subject, (ii) the subject is at increased risk of recurrence of a cancer, including a shorter time to recurrence, or of an earlier or imminent recurrence (iii) the subject had a cancer prior to the testing and developed an immune response to the peptide.

The peptide can be one that is a peptide of a protein correlated with a cancer, such as a peptide of an oncogenic protein. In one embodiment, the peptide is to Her2/neu, such as GP2.

Terms used herein will be understood to take on their ordinary meaning in the relevant art unless specified otherwise. Several terms used herein, and their meanings are set forth below.

“Relapse” and “recurrence” are used interchangeably herein, and refer to the diagnosis of return, or signs and symptoms of return of breast cancer after a period of improvement or remission. Cancer recurrence can be diagnosed using standard of care methods such as patient follow-up visits, blood test, mammogram, and imaging.

As used herein, “oncoprotein” and “oncogenic protein” are used interchangeably and refer to a protein that is correlated to cancer in a human. Oncoproteins are encoded by oncogenes. Oncogenes are the mutated forms of normal cellular genes (proto-oncogenes). The protein products of proto-oncogenes stimulate cell division and/or inhibit cell death. Proto-oncogenes can be likened to the gas pedal in a car. Normally, internal and external signals strictly regulate the activity of the proto-oncogenes, but oncogenes are defective and are ‘on’ even when they do not receive appropriate signals. Oncogenes also help cells to ignore negative signals that would prevent a healthy cell from dividing. Oncogenes can cause cells to divide continuously even in the absence of any pro-growth signals. The following list describes different cellular roles for a few of the many known oncogenes.

HER-2/neu: HER-2/neu encodes for a cell surface receptor that can stimulate cell division, the HER-2/neu gene is amplified in up to 30% of human breast cancers; RAS: the Ras gene products are involved in kinase signaling pathways that ultimately control transcription of genes, regulating cell growth and differentiation, overexpression and amplification of RAS can lead to continuous cell proliferation; MYC: the Myc protein is a transcription factor and controls expression of several genes, Myc is thought to be involved in avoiding the cell death mechanism, MYC oncogenes may be activated by gene rearrangement or amplification; SRC: SRC was the first oncogene discovered, the Src protein is a tyrosine kinase which regulates cell activity; hTERT: hTERT codes for an enzyme (telomerase) that maintains chromosome ends, in most normal cells telomerase is only present during fetal development, activation of hTERT in adult cells gives them the ability to divide indefinitely; BCL-2: the Bcl-2 protein works to prevent cell death (apoptosis), overexpression of BCL-2 allows continued division of mutated cells. An oncogenic protein can be one that is expressed by non-cancer cells but at higher levels in cancer cells, or one that is not expressed in non-cancer cells but expressed in cancer cells. Examples of oncogenic proteins are shown in Table 1 (also on the world wide web at cancerquest.org/cancer-biology/cancer-genes.

TABLE 1

| Oncogene | Function/Activation | Cancer* |
|---|---|---|
| ABL₁ | Promotes cell growth through | Chronic myelogenous leukemia |
| | tyrosine kinase activity | |
| AFF₄/MLLT₁₁ | Fusion affects the MLLT₁₁ | Acute leukemias |
| | transcription | |
| | factor/methyltransferase. MLLT₁₁ | |
| | is also called HRX, ALL₁ and | |
| | HTRX₁ | |
| AKT₂ | Encodes a protein- | Ovarian cancer |
| | serine/threonine kinase | |
| ALK | Encodes a receptor tyrosine | Lymphomas |
| | kinase | |
| ALK/NPM | Translocation creates fusion | Large cell lymphomas |
| | protein with | |
| | nucleophosmin(npm) | |
| RUNX₁ (AML₁) | Encodes a transcription factor | Acute myeloid leukemia |
| RUNX₁/MTG8(ETO) | New fusion protein created by | Acute leukemias |
| | translocation | |
| AXL | Encodes a receptor tyrosine | Hematopoietic cancers |
| | kinase | |
| BCL-2, 3, 6 | Block apoptosis (programmed | B-cell lymphomas and |
| | cell death) | leukemias |
| BCR/ABL | New protein created by fusion of | Chronic myelogenous and acute |
| | bcr and abl triggers unregulated | lymphotic leukemia |
| | cell growth | |
| MYC (c-MYC) | Transcription factor that | Leukemia; breast, stomach, |
| | promotes cell proliferation and | lung, cervical, and colon |
| | DNA synthesis | carcinomas; neuroblastomas |
| | | and glioblastomas |
| MCF₂ (DBL) | Guanine nucleotide exchange | Diffuse B-cell lymphoma |
| | factor | |
| DEK/NUP₂₁₄ | New protein created by fusion | Acute myeloid leukemia |
| TCF₃/PBX₁ | New protein created by fusion | Acute pre B-cell leukemia; TCF₃ |
| | | also called E₂A |
| EGFR | Cell surface receptor that | Squamous cell carcinoma, |
| | triggers cell growth through | glioblastomas, lung cancer |
| | tyrosine kinase activity | |
| MLLT₁₁ | Fusion protein created by a | Acute leukemias |
| | translocation t(11; 19). | |
| ERG/FUS | Fusion protein created by t(16:21) | Myeloid leukemia |
| | translocation. The erg protein is | |
| | a transcription factor. | |
| ERBB₂ | Cell surface receptor that | Breast, salivary gland, and |
| | triggers cell growth through | ovarian carcinomas |
| | tyrosine kinase activity; also | |
| | known as HER₂ or neu | |
| ETS₁ | Transcription factor | Lymphoma |
| EWSR₁/FLI₁ | Fusion protein created by t(11:22) | Ewing Sarcoma |
| | translocation. | |
| CSF₁R | Tyrosine kinase | Sarcoma |
| FOS | Transcription factor for API | Osteosarcoma |
| FES | Tyrosine kinase | Sarcoma |
| GLI₁ | Transcription factor | Glioblastoma |
| GNAS (GSP) | Membrane associated G protein | Thyroid carcinoma |
| HER₂/neu | Overexpression of signaling | Breast, ovarian, gastric, and |
| | kinase due to gene amplification | cervical carcinomas |
| TLX₁ | Transcription factor; aka HOX₁₁ | Acute T-cell leukemia |
| FGF₄ | Encodes fibroblast growth | Breast and squamous cell |
| | factor; aka HST₁ | carcinomas |
| IL₃ | Cell signaling molecule | Acute pre B-cell leukemia |
| FGF₃ (INT-₂) | Encodes a fibroblast growth | Breast and squamous cell |
| | factor | carcinomas |
| JUN | Transcription factor for API | Sarcoma |
| KIT | Tyrosine kinase | Sarcoma |
| FGF₄ (KS₃) | Herpes virus encoded growth | Kaposi's sarcoma |
| | factor | |
| K-SAM | Fibroblast growth factor | Stomach carcinomas |
| | receptor | |
| AKAP₁₃ | Guanine nucleotide exchange | Myeloid leukemias |
| | factor; aka LBC | |
| LCK | Tyrosine kinase | T-cell lymphoma |
| LMO₁, LMO₂ | Transcription factors | T-cell lymphoma |
| MYCL | Transcription factor | Lung carcinomas |
| LYL₁ | Transcription factor | Acute T-cell leukemia |
| NFKB₂ | Transcription factor. Also | B-cell lymphoma |
| | called LYT-₁₀ | |
| NFKB₂/Cα₁ | Fusion protein formed by | |
| | the (10; 14)(q24; q32) translocation | |
| | of NFKB₂ next to the C alpha₁ | |
| | immunoglobulin locus. | |
| MAS₁ | Angiotensin receptor | Mammary carcinoma |
| MDM₂ | Encodes a protein that inhibits | Sarcomas |
| | and leads to the degradation of | |
| | p53 | |
| MLLT₁₁ | Transcription | Acute myeloid leukemia |
| | factor/methyltransferase (also | |
| | called HRX and ALL₁) | |
| MOS | Serine/threonine kinase | Lung cancer |
| RUNX₁T₁ | Fusion of transcription repressor | Acute leukemias |
| | to factor to a transcription | |
| | factor. Also known as MTG8 and | |
| | AML₁-MTG8 | |
| MYB | Transcription factor | Colon carcinoma and leukemias |
| MYH₁₁/CBFB | New protein created by fusion of | Acute myeloid leukemia |
| | transcription factors via an | |
| | inversion in chromosome 16. | |
| NEU | Tyrosine kinase. Also called | Glioblastomas, and squamous |
| | ERBB₂ or HER₂ | cell carcinomas |
| MYCN | Cell proliferation and DNA | Neuroblastomas, |
| | synthesis | retinoblastomas, and lung |
| | | carcinomas |
| MCF₂L (OST) | Guanine nucleotide exchange | Osteosarcomas |
| | factor | |
| PAX-₅ | Transcription factor | Lympho-plasmacytoid B-cell |
| | | Lymphoma |
| PBX₁/E₂A | Fusion protein formed via t(1:19) | Acute pre B-cell leukemia |
| | translocation. Transcription | |
| | factor | |
| PIM₁ | Serine/threonine kinase | T-cell lymphoma |
| CCND₁ | Encodes cyclin D₁. Involved in | Breast and squamous cell |
| | cell cycle regulation. Also called | carcinomas |
| | PRAD₁ | |
| RAF₁ | Serine/threonine kinase | Many cancer types |
| RARA/PML | Fusion protein caused by t(15:17) | Acute premyelocytic leukemia |
| | translocation. Retinoic acid | |
| | receptor. | |
| HRAS | G-protein. Signal transduction. | Bladder carcinoma |
| KRAS | G-protein. Signal transduction | Lung, ovarian, and bladder |
| | | carcinoma |
| NRAS | G-protein. Signal transduction | Breast carcinoma |
| REL/NRG | Fusion protein formed by deletion | B-cell lymphoma |
| | in chromosome 2. Transcription | |
| | factor. | |
| RET | Cell surface receptor. Tyrosine | Thyroid carcinomas, multiple |
| | kinase | endocrine neoplasia type 2 |
| RHOM₁, RHOM₂ | Transcription factors aka LMO₁ | Acute T-cell leukemia |
| | and LMO₂ | |
| ROS₁ | Tyrosine kinase | Sarcoma |
| SKI | Transcription factor | Carcinomas |
| SIS (aka PDGFB) | Growth factor | Glioma, fibrosarcoma |
| SET/CAN | Fusion protein formed by | Acute myeloid leukemia |
| | rearrangement of chromosome | |
| | 9. Protein localization | |
| SRC | Tyrosine kinase | Sarcomas |
| TAL₁, TAL₂ | Transcription factor TAL₁ is also | Acute T-cell leukemia |
| | called SCL | |
| NOTCH₁ (TAN₁) | Altered form of Notch (a cellular | Acute T-cell leukemia |
| | receptor) formed by t(7:9) | |
| | translocation | |
| TIAM₁ | Guanine nucleotide exchange | T-lymphoma |
| | factor | |
| TSC₂ | GTPase activator | Renal and brain tumors |
| NTRK₁ | Receptor tyrosine kinase | Colon and thyroid carcinomas |

*The cancer types listed in this column are those that are predominantly associated with each oncogene but this is not a complete list.

As used herein, a “peptide” refers to a portion of protein, such as a protein that correlates with a cancer, e.g., an oncoprotein, that is of a size sufficient to generate an immune response. A peptide can be at least 6 amino acids, at least 9 amino acids, or at least 12 amino acids in length.

Unless otherwise specified, “a,” “an,” “the,” and “at least one” are used interchangeably and mean one or more than one.

As used in this specification and the appended claims, the term “or” is generally employed in its sense including “and/or” unless the content clearly dictates otherwise. The term “and/or” means one or all of the listed elements or a combination of any two or more of the listed elements. The use of “and/or” in some instances does not imply that the use of “or” in other instances may not mean “and/or.”

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure.

As used herein, “have,” “has,” “having,” “include,” “includes,” “including,” “comprise,” “comprises,” “comprising” or the like are used in their open ended inclusive sense, and generally mean “include, but not limited to,” “includes, but not limited to,” or “including, but not limited to.”

It is understood that wherever embodiments are described herein with the language “have,” “has,” “having,” “include,” “includes,” “including,” “comprise,” “comprises,” “comprising” and the like, otherwise analogous embodiments described in terms of “consisting of” and/or “consisting essentially of” are also provided. The term “consisting of” means including, and limited to, whatever follows the phrase “consisting of.” That is, “consisting of” indicates that the listed elements are required or mandatory, and that no other elements may be present. The term “consisting essentially of” indicates that any elements listed after the phrase are included, and that other elements than those listed may be included provided that those elements do not interfere with or contribute to the activity or action specified in the disclosure for the listed elements.

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments.

Throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

In the description herein particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The above summary of the present disclosure is not intended to describe each disclosed embodiment or every implementation of the present disclosure. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

The following detailed description of illustrative embodiments of the present disclosure may be best understood when read in conjunction with the following drawings.

FIG. 1 shows the time to recurrence in patients experiencing breast cancer recurrence by those patients who have a baseline immune response to GP2 and those who do not. Analysis of the time to recurrence among those recurring found that the median time to recurrence was 0.6 years for those with a baseline response while those that did not have a positive baseline DTH response to GP2 took 1.2 years to recur. The number at risk for “Baseline Response” and “None” refer to the number of patients with that type of response at each tested timepoint.

FIG. 2 shows that baseline DTH response is predictive of disease-free survival in a pooled population of HER2 positive and HER2 low patients.

FIG. 3 shows correlation between month 6 DTH and injection site reaction at dose 6 injection in GLSI-100 treated subjects. A single outlier was excluded from this analysis due to the influence of this point.

FIG. 4 shows correlation between baseline DTH and injection site reaction at dose 1 injection in GLSI-100 treated subjects.

### DETAILED DESCRIPTION

The inventors have determined that the presence of an immune response to a peptide, such as a peptide of an oncogenic protein, correlated with a cancer in a human is prognostic for the state of that cancer in a subject. The presence of an immune response can indicate that a cancer is present in the subject, that the subject is at increased risk of recurrence for a cancer, including a shorter time to recurrence, or of an earlier or imminent recurrence, or the subject had a cancer prior to testing for an immune response and developed an immune response to an oncoprotein, or a combination thereof. Examples of cancers that can be analyzed using the methods described herein include Her2/neu positive cancers such as breast cancer, ovarian cancer, gastric cancer and many other cancers, and may include many different types of proteins, such as oncoproteins, or combinations thereof. Other examples of cancers that can be analyzed using the methods described herein include, but are not limited to, the cancers listed in Table 1. The oncogenic proteins that correlate with different cancers are encoded by the oncogenes listed in Table 1. The peptide can be any set of consecutive amino acids from a peptide, such as a peptide that correlates with a cancer, e.g., an oncogenic protein. In one embodiment, the peptide is one that has therapeutic activity by activating the immune system, e.g., the peptide can be used to treat the subject to increase the immune response against a cancer and thus reduce an aspect of cancer and improve the health of the subject. For instance, a peptide can be one that reduces the recurrence of cancer in the subject by training the immune system to attack the cancer, including a recurrence caused by metastatic cancer. The therapeutic activity can result from the peptide or from the combination of the peptide with one or more other compounds. An example of a peptide with therapeutic activity is GP2, which is typically administered with GMCSF.

The methods include determining a subject's immune response to a peptide, such as a protein correlated to a cancer, e.g., a peptide of an oncogenic protein. The methods include a subject that is a human, or an animal typically used as a model system for evaluating cancer-related treatments. In one embodiment, the subject is a human. In one embodiment, the subject can be a person that is at risk for a cancer, and the peptide used in the method is from an oncogenic protein that correlates with that cancer. A person that is at risk for a cancer includes, but is not limited to, a person who has been treated for a cancer and is in complete remission or in partial remission. A person that is at risk for a cancer also includes, but is not limited to, person having one or more biomarkers, or a person with genetic risk for a cancer. Biomarkers are molecules, such as a gene or a protein, present in a subject and indicative of the progression of cancer. Examples of biomarkers include BRCA1, BRCA2, and prostate specific antigen (PSA). An example of a person with a genetic risk is one with a history of a cancer in relatives, such as first-degree relatives. In one embodiment, the subject can be one that has not previously been diagnosed as having a cancer. Such a person can be one that is at risk for a cancer, for instance, the subject can be one that has not been diagnosed as having a cancer but has one or more biomarkers and/or has a genetic risk of a cancer.

In another embodiment, the subject can be a person that was previously treated for a cancer, and the peptide used in the method is from a protein, such as a protein, for instance an oncogenic protein, that correlates with that cancer. In one embodiment, the subject is one that was previously treated for a cancer that was HER2/neu positive at a low, intermediate, or high level. HER2-positive cancers, such as breast cancers, are typically evaluated for HER2/neu expression and assigned an immunohistochemistry (IHC) score of 0 or 1+ which can be referred to as HER2/neu low, 2+ which can be referred to as HER2/neu intermediate, or 3+ which can be referred to as HER2/neu high. Methods for evaluating HER2/neu expression and assigning an IHC score are known in the art. In this embodiment, the cancer can be, but is not limited to, HER2/neu positive breast cancer, HER2/neu positive ovarian cancer, or HER2/neu positive gastric cancer, or any other HER2/neu positive cancer. In one embodiment, the cancer is HER2/neu positive breast cancer.

In one embodiment, the method can be used to determine an immune response to a peptide from a Her2/neu protein. The peptide can be from the cytoplasmic domain, the extracellular domain, or the transmembrane domain of a Her2/neu protein. In one embodiment, the peptide is from the transmembrane domain, and a non-limiting example of a peptide from the transmembrane domain is GP2 (Ile Ile Ser Ala Val Val Gly Ile Leu, SEQ ID NO:1). GP2, in combination with granulocyte-macrophage colony-stimulating factor (GM-CSF), is a therapy used to reduce cancer recurrence in breast cancer patients with tumors expressing any degree of HER2/neu (Patel et al., Five year median follow-up data from a prospective, randomized, placebo-controlled, single-blinded, multicenter, Phase IIb study evaluating the reduction of recurrences using HER2/neu peptide GP2+GM-CSF vs. GM-CSF alone after adjuvant trastuzumab in HER2 positive women with operable breast cancer. Presented at: 2020 San Antonio Breast Cancer Symposium; Dec. 8-11, 2020.

The immune response to a peptide, such as a peptide of a protein that correlates with a cancer, for instance an oncogenic protein, can be determined by any method. Immune response can be measured by, but is not limited to, delayed type hypersensitivity (DTH), binding assays to T cells, functional cell-based assays for cytokine secretion such as Elispot, including an Elispot assay that quantifies T cell viability, measurement of antibody titers, and T cell assays including identification of specific T cells against any cancer target and including binding assays of T cells to HLA-peptide reagents that quantify peptide specific T cells. An immune response is considered a positive immune response if it is greater than the immune response of a suitable negative control. Thus, any increase in an immune response that is greater than a suitable negative control is a positive immune response. The skilled person can readily determine a negative control that can be used to determine if an immune response to a peptide, such as a peptide that correlates with a cancer, for instance an oncogenic protein. In one embodiment, the immune response is measured by testing a subject's DTH response to an intradermal injection of a peptide, such as a peptide that correlates with a cancer, for instance the protein at lower, similar, or higher quantities than a treatment dose. Methods for testing a DTH response are known in the art and routine. In one embodiment, induration (e.g., localized hardening) of the skin at the administration site is measured. When induration is determined the perpendicular diameter of the induration is typically measured. Measurement can be by any suitable method, including calipers, ruler, or the ball point pen technique. The measurement can be expressed as the largest diameter of the skin reaction, or the orthogonal mean of the diameters is determined. An induration measurement of at least 3 mm, at least 5 mm, at least 7 mm or at least 10 mm or higher can be considered a positive DTH response. The DTH response of a subject can be measured from 24 to 72 hours, typically from 48 hours to 72 hours, after administration of the peptide at the chosen site.

In those embodiments where the peptide is one that has therapeutic activity, the peptide can be used to determine an immune response before the peptide has been used for therapy. For instance, the peptide can be used to determine an immune response after the subject is diagnosed as having cancer, or after the subject has been treated, for instance by surgery or chemotherapy, for cancer. In one embodiment, GP2 is used as the peptide and it is administered for determining an immune response before the GP2 is used therapeutically. In another embodiment, GP2 is used as the peptide and it is administered for determining an immune response at approximately the same time the GP2 is administered therapeutically. Typically, the immune response that results from a test administered prior to the commencement of treatment or at approximately the same time as the commencement of treatment (e.g., before a subject can mount an immune response to an administered peptide) is referred to herein as a “baseline” response.

In those embodiments where the peptide is one that is not therapeutic, the peptide can be used to determine an immune response at any time, such as before the subject is diagnosed as having cancer, after the subject is diagnosed as having cancer, or after the subject has been treated, for instance by surgery or chemotherapy, for a cancer.

The amount of peptide administered to a subject to measure an immune response can vary and is not intended to be limiting. For instance, the range can be from 0.01 milligram/milliliter (mg/ml) to 1 mg/ml. When the peptide is a Her2/neu peptide, such as GP2, the amount can be 0.05 mg/ml to 0.4 mg/ml, and in one embodiment is 0.2 mg/ml. In one embodiment, the composition of peptide administered does not include an immunomodulatory agent such as GMCSF. The volume used can vary and is not intended to be limiting. For instance, the volume can be from 0.8 ml to 0.2 ml, and in one embodiment is 0.5 ml. The skilled person will recognize that a saline injection can be used as a negative control.

When determining if a subject has a positive immune response to a peptide, such as a peptide that correlates with a cancer, for instance an oncogenic protein, the immune response to the peptide can be tested before baseline, at baseline, or after baseline. Testing before baseline refers to a time before the subject begins treatment. For instance, the peptide is delivered to the subject before the subject begins treatment with a peptide. Testing before baseline can occur at any time before treatment begins, such as at least 6 months, at least 5 months, at least 4 months, at least 3 months, at least 2 months, or at least 1 month, before treatment begins, or at least 20, at least 10, at least 9, at least 8, at least 7, at least 6, at least 5, at least 4, at least 3, at least 2, or at least 1 day before treatment begins.

Testing at baseline refers to a time before the subject has mounted an immune response to a peptide administered to treat the subject. For instance, the peptide is delivered to the subject for testing an immune response at approximately the same time the peptide is administered to treat the subject, or the peptide is delivered after the peptide is administered to treat the subject but before the subject has mounted an immune response to a peptide administered to treat the subject. When the testing is at baseline the peptide is delivered to the subject at approximately the same day or same time the peptide is administered to treat the subject, or within 1, 2, 3, 4, 5, 6, 7, 8, 9 or 10 days after the peptide is administered to treat the subject.

Testing after baseline refers to a time after the subject has had the opportunity to mount an immune response to a peptide administered to treat the subject. For instance, the peptide is delivered to the subject for testing an immune response at least 11 days after the peptide is administered to treat the subject. In some embodiments, testing after baseline is accomplished by evaluating the immune response resulting from an injection site reaction.

Evaluating the immune response resulting from an injection site reaction can be done in lieu of using a separate delivery site or can be done to complement the use of a separate delivery site. Evaluating the immune response resulting from an injection site reaction can be done by measuring the largest perpendicular diameters and the resulting orthogonal mean. In seme embodiments the immune response can be determined by any method of measuring the injection site reaction. Thus, in some embodiments, the immune response is measured by testing a subject's DTH response at an injection site where the peptide was administered to treat the subject.

Baseline GP2 immune response as measured by the DTH test was demonstrated to be an independent prognostic factor for recurrence. Thus, in one embodiment, methods of the present disclosure can include determining whether the subject is at a higher risk of recurrence of cancer. A positive immune response before baseline, at baseline, or after baseline is associated with an increased likelihood of recurrence and/or a recurrence resulting earlier than not having a positive immune response. In one embodiment, a positive immune response to a peptide of an oncogenic protein such as GP2 is indicative of a reduction in time to recurrence of at least 40% to no greater than 60%, at least 45% to no greater than 55%, or 50% compared to the time to recurrence of a subject that does not have a positive immune response. Accordingly, a clinician can be better informed of which subjects may require increased surveillance and/or care.

The inventors have determined that a subject having a positive baseline DTH response to GP2 has an increased risk of experiencing a breast cancer recurrence compared to those not having a baseline DTH response to GP2. As described in Example 1, the DTH response of 180 subjects was evaluated. The subjects had node-positive and high-risk node-negative breast cancers that were HER2 3+ positive or low HER2 expressors (1-2+). The breast cancer had been surgically treated, and the subjects had been treated with trastuzumab (Herceptin®). A positive baseline DTH response occurred in 22.8% of the subjects, and of all the subjects having a later breast cancer recurrence, 36.4% had a positive baseline response. Analysis of the time to recurrence among those recurring found that the median time to recurrence was 0.6 years for those with a baseline response while those that did not have a positive baseline DTH response to GP2 took 1.2 years to recur. Thus, in one embodiment a positive DTH response at baseline can be indicative of whether the subject is more likely to have a recurrence of a cancer, and the cancer that is likely to relapse is a HER2/neu cancer. In another embodiment, a positive DTH response at baseline can be used to predict whether a cancer may be present in the subject before other routine detection methods are able to identify the presence of cancer cells. The cancer cells that may be present are HER2/neu-positive cancer cells.

Following a positive immune response to a peptide, a subject can be treated with a therapy to reduce the cancer present, reduce the risk of recurrence of the cancer, or a combination thereof. The therapy can include, but is not limited to, antibody, antibody drug conjugate, checkpoint inhibitor, hormone therapy, tyrosine kinase inhibition, surgery, chemotherapy, or radiation therapy.

In one embodiment, the presence of a positive DTH response at baseline in a subject can be used to predict the response to a GP2-based therapy in a subject. In one embodiment, the presence of a positive baseline DTH response can be used by the skilled person to determine that GP2-based therapy may be unnecessary due to existing GP2 specific immunity and optionally replaced with a different therapy.

The inventors have determined that GLSI-100 safely elicited a potent immune response, as evidenced by injection site reactions (ISRs) that correlate to and may complement or replace DTH tests or other immune response methods.

The invention is defined in the claims. However, below there is provided a non-exhaustive listing of non-limiting exemplary aspects. Any one or more of the features of these aspects may be combined with any one or more features of another example, embodiment, or aspect described herein.

### Exemplary Aspects

Aspect 1 is a method comprising: testing a subject's immune response to a peptide of a protein, wherein the subject had a cancer; determining that the subject has a positive immune response to a peptide, wherein the testing occurs at baseline, after baseline, or before baseline, wherein the positive immune response indicates the subject is at increased risk of recurrence of a cancer, a cancer is present in the subject, the subject had a cancer prior to the testing and developed an immune response to the peptide, or a combination thereof, or the subject has developed an immune response to the peptide for any other reason.

Aspect 2 is a method for determining time to recurrence of a cancer, comprising: testing a subject's immune response to a peptide of a protein correlating with a cancer, wherein the subject had the cancer; determining that the subject has a positive immune response to the peptide, wherein the presence of an immune response to the peptide is indicative of a shorter time to recurrence of the cancer than the absence of the response.

Aspect 3 is a method for identifying the presence of a cancer, comprising: testing a subject's immune response to a peptide of a protein correlating with a cancer; determining that the subject has a positive immune response to the peptide related to a current cancer that the immune system is responding to and thus generating an immune response to the peptide.

Aspect 4 is the method of any preceding aspect, wherein the peptide is at least 6 amino acids.

Aspect 5 is the method of any preceding aspect, wherein the peptide is a peptide of an oncogenic protein.

Aspect 6 is the method of any preceding aspect, wherein the oncogenic protein is HER2/neu.

Aspect 7 is the method of any preceding aspect, wherein the peptide is GP2.

Aspect 8 is the method of any preceding aspect, wherein the cancer comprises HER2/neu expressing cells.

Aspect 9 is the method of any preceding aspect, wherein the HER2/neu expressing cells are HER2/neu 3+, HER2/neu positive, HER2/neu intermediate, or HER2/neu low.

Aspect 10 is the method of any preceding aspect, wherein the subject had or has breast cancer.

Aspect 11 is the method of any preceding aspect, wherein the positive immune response indicates a cancer is present in the subject or the cancer for which the subject is at increased risk of recurrence is breast, and wherein the cancer is metastatic cancer.

Aspect 12 is the method of any preceding aspect, wherein the testing comprises a delayed type hypersensitivity (DTH) immune response to the peptide.

Aspect 13 is the method of any preceding aspect, wherein the peptide is a peptide of an oncogenic protein.

Aspect 14 is the method of any preceding aspect, wherein the oncogenic protein is HER2/neu.

Aspect 15 is the method of any preceding aspect, wherein the peptide of the oncogenic protein is GP2.

Aspect 16 is the method of any preceding aspect, wherein the subject had or has breast cancer, ovarian cancer, gastric cancer, cervical cancer, or any other HER2/neu expressing cancer.

Aspect 17 is the method of any preceding aspect, wherein a positive DTH immune response is indicative of HER2/neu-expressing cancer cells in the subject, increased risk of recurrence of a HER2/neu-expressing cancer in the subject, the subject had a HER2/neu-expressing cancer prior to the testing, or a combination thereof.

Aspect 18 is the method of any preceding aspect, wherein the subject is undergoing treatment with a therapeutic peptide against a cancer, and wherein the testing comprises evaluation or measurement of an injection site reaction.

Aspect 19 is the method of any preceding aspect, wherein the testing comprises measuring the largest perpendicular diameters and the resulting orthogonal mean.

Aspect 20 is the method of any preceding aspect, wherein the peptide is a peptide of an oncogenic protein.

Aspect 21 is the method of any preceding aspect, wherein the oncogenic protein is HER2/neu.

Aspect 22 is the method of any preceding aspect, wherein the peptide of the oncogenic protein is GP2.

Aspect 23 is the method of any preceding aspect, wherein the subject had breast cancer, ovarian cancer, gastric cancer, cervical cancer, or any other HER2/neu expressing cancer.

Aspect 24 is the method any preceding aspect, wherein a positive DTH immune response is indicative of HER2/neu-expressing cancer cells in the subject, increased risk of recurrence of a HER2/neu-expressing cancer in the subject, the subject had a HER2/neu-expressing cancer prior to the testing, or a combination thereof.

Aspect 25 is the method of any preceding aspect, wherein the positive DTH immune response comprises an induration.

Aspect 26 is the method of any preceding aspect, wherein the positive DTH immune response comprises an induration.

Aspect 27 is the method of any preceding aspect, wherein the subject has a cancer, the method further comprising treating the cancer.

Aspect 28 is the method of any preceding aspect, wherein the subject has an increased risk of recurrence of cancer or a shorter time to recurrence of cancer, the method further comprising treating the subject to reduce, prevent, or stop the recurrence of cancer.

Aspect 29 is the method of any preceding aspect, wherein the lack of a positive immune response to the peptide is indicative of the absence of cancer or a reduced risk of recurrence of a cancer.

Aspect 30 is the method of any preceding aspect, wherein the testing comprises an immune response assay that can detect a subject's immune response to a peptide.

Aspect 31 is the method of any preceding aspect, wherein the immune response assay is chosen from a DTH reaction, an ELISPOT assay, an antibody titer assay, and a T cell assay.

Aspect 32 is the method of any preceding aspect, wherein the testing compromises measurement of an injection site reaction to a therapeutic peptide against a cancer.

Aspect 33 is the method of any preceding aspect, wherein the time to recurrence is reduced by at least 40% to no greater than 60% compared to a subject that had the cancer and did not have an immune response to the peptide.

Aspect 34 is the method of any preceding aspect, wherein the subject has not been diagnosed as having a cancer before the testing.

### EXAMPLES

The present disclosure is illustrated by the following examples. It is to be understood that the particular examples, materials, amounts, and procedures are to be interpreted broadly in accordance with the scope and spirit of the disclosure as set forth herein.

Background for Examples 1-5: Delayed type hypersensitivity (DTH) skin tests in the randomized, active-controlled, single-blinded, multicenter Phase IIb trial investigating GLSI-100 (GP2+GM-CSF) administered in the adjuvant setting to node-positive and high-risk node-negative breast cancer patients with tumors expressing any degree of HER2 (immuno-histochemistry [IHC] 1-3+) (NCT00524277) were analyzed. The trial enrolled HLA-A*02 patients randomized to receive GLSI-100 versus GM-CSF alone. The trial's primary objective was to determine if treatment with GLSI-100, a HER2-derived peptide, reduces recurrence rates.

### Example 1

Immune response analysis at baseline in phase IIb trial of GP2

Methods: Consented patients were randomized and scheduled to receive GLSI-100 (500 microgram (mcg) GP2: 125 mcg GM-CSF) or control (GM-CSF only) via 6 intradermal injections every 3-4 weeks as part of the Primary Immunization Series (PIS) for the first 6 months and 4 booster intradermal injections every 6 months thereafter. Boosters were introduced during the trial, thus some patients did not receive all 4 boosters. DTH skin tests were assessed at baseline and after the 6^{th} dose with the orthogonal mean of each skin reaction measured 48-72 hours after injection using the sensitive ballpoint-pen method.

Results: The study enrolled 180 patients across 16 clinical sites with both HER2 3+ positive and low HER2 expressors (1-2+). After 5 years of follow-up, the Kaplan-Meier estimated 5-year disease-free survival (DFS) rate in the 46 HER2 3+ patients treated with GLSI-100, if the patient was followed for 6 months or more and remained disease-free over the first 6 months, was 100% versus 89.4% (95% CI:76.2, 95.5%) in the 50 placebo patients treated with GM-CSF (p=0.0338). GLSI-100 was shown to be well tolerated with no SAEs deemed related to study medication and elicited a potent immune response measured by local skin tests and immunological assays. Injection site reactions were common, occurring in almost 100% of patients treated with either GLSI-100 or GM-CSF alone. Previous publications have reported the increase in DTH response reported among patients after treatment with GLSI-100.

It was of interest to understand the positive DTH responses to GP2 noted at baseline. 22.8% of patients reacted to GP2 at baseline with induration of 5 mm or greater. In the subgroup of patients who later experienced a breast cancer recurrence, 36.4% (8/22) had such a baseline response. Analysis of the time to recurrence among those recurring found that the median time to recurrence was 0.6 years for those with a baseline response while those that did not have a positive baseline DTH response to GP2 took 1.2 years to recur.

Conclusions: This study demonstrated that GLSI-100 safely elicited a potent immune response as evidenced by increased DTH skin responses with treatment paired with improved disease-free survival. It is theorized that a positive baseline DTH skin test to GP2 may be evidence of an existing immune response to GP2 associated with residual disease, impending recurrence, or prior treatments. Further studies assessing if GP2 immune response is an important prognosticator of cancer disease state or recurrence are planned.

### Example 2

This trial explored HER2 3+ patients, who received a standard course of trastuzumab after surgery, and HER2 1-2+ patients, who did not receive trastuzumab after surgery. A DTH reaction was used to assess in vivo immune responses in patients (n=150) prior to exposure to study medication and after 6 months of the first dose. The DTH orthogonal mean was measured 48-72 hours after injection using the sensitive ballpoint-pen method. DTH reactions after the PIS were greater compared to baseline DTH reactions. In addition, the DTH reactions after the PIS were significantly greater in GLSI-100 treated patients than in control patients (10.8 mm vs. 0.0 mm, p=0.009).

It was noted that some patients reported significant DTH responses at baseline, prior to exposure to GP2. It was found that 22.8% of patients reacted to GP2 at baseline with induration of 5 mm of greater in the baseline DTH test. A positive response was defined as a site measurement of 5 mm of larger.

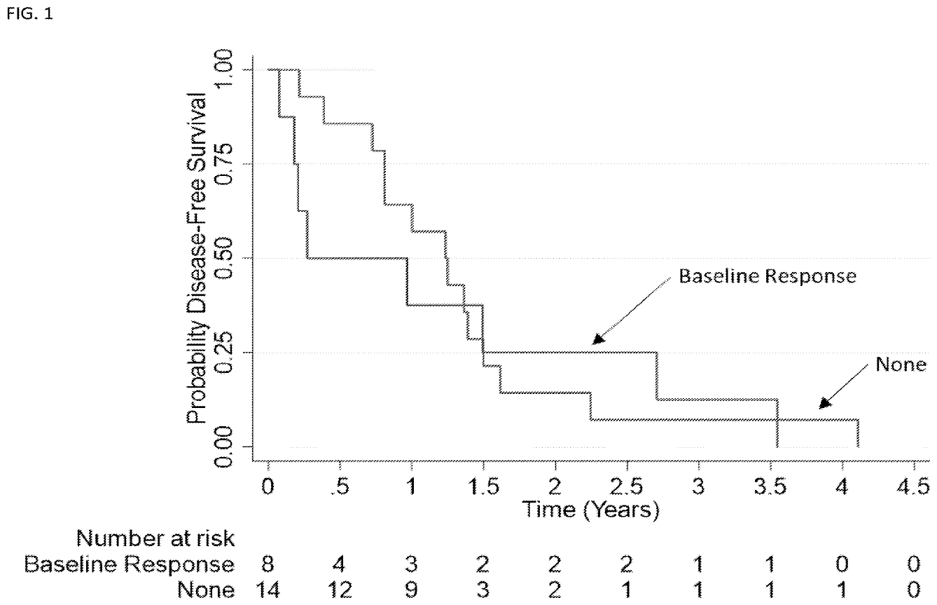

A Kaplan-Meir analysis in the subgroup of patients that experienced a recurrence of breast cancer over the course of the study investigated time to recurrence by baseline DTH response (positive=Baseline Response/negative=None). As shown in FIG. 1, although the survival curves become quite similar after 1.5 years, which makes the two curves statistically the same, p=0.7140, the early effect of baseline response to GP2 assessed by DTH creates a readily apparent effect on early recurrence. The median time to recurrence of those with a baseline DTH response is 99 days (0.27 years) while those with no baseline DTH took 438 days (1.2 years) to recur by life table analysis techniques. Those summary statistics by standard methods are 0.6 years for those with a baseline response and 1.2 years for those with none.

Note, these analyses were conducted independent of the treatment group assigned as the interest was how baseline DTH results might predict early recurrence. Table 2 lists the raw data for the subjects who recurred over the course of follow-up and their baseline and 6 month DTH assessments.

TABLE 2
Data Listing of Baseline and 6 Month DTH in Patients Experiencing Breast Cancer Recurrence

| | | | | Tumor | | Time to | Baseline | 6 Month |
|---|---|---|---|---|---|---|---|---|
| | | Node | | Size | HER2 | Recurrence | DTH | DTH |
| Patient | Treatment | Status | T Stage | (cm) | Status | (days) | (mm) | (mm) |
| PG66V66 | GP2 | N1a | T1 | 2.0 | 1 | 76 | 43 | |
| AG45V45 | Placebo | N3 | T2 | 2.1 | 2 | 353 | 29 | 44 |
| PG23V23 | GP2 | N1a | T3 | 5.0 | 1 | 1295 | 13 | 25 |
| AG8V8 | Placebo | N0 | T1a | 1.3 | 2 | 988 | 10 | 30 |
| AG1V1 | Placebo | N2a | T1a | 1.4 | 2 | 545 | 9 | 34 |
| BR-02 | GP2 | N3a | T2 | 2.5 | 3 | 28 | 9 | |
| PG47V47 | GP2 | N1a | T1 | 0.7 | 2 | 99 | 8 | |
| PG25V25 | GP2 | N1a | ypT0 | 0.0 | 3 | 66 | 7 | |
| PG4V4 | GP2 | i+ | T2 | 2.3 | 1 | 820 | 0 | 24 |
| PG2V2 | GP2 | N0 | T2 | 4.5 | 1 | 265 | 0 | 20 |
| PG9V9 | GP2 | N1a | T4 | 1.5 | 1 | 498 | 0 | 16 |
| BR-103 | GP2 | N3a | T1 | 2.0 | 2 | 507 | 0 | 15 |
| AG26V26 | Placebo | N2a | T2 | 2.4 | 2 | 1500 | 0 | 6 |
| BR-119 | Placebo | N1a | T1 | 0.9 | 3 | 451 | 0 | 6 |
| BR-109 | GP2 | N3a | T3 | 5.5 | 1 | 366 | 0 | 3 |
| AG28V28 | Placebo | N0 | T2 | 2.2 | 1 | 456 | 0 | 0 |
| AG39V39 | Placebo | N2a | T3 | 9.0 | 1 | 296 | 0 | 0 |
| AG3V3 | Placebo | N0 | T2 | 2.3 | 1 | 296 | 0 | 0 |
| PG42V42 | GP2 | N0 | T2 | 2.2 | 2 | 590 | 0 | 0 |
| BR-129 | GP2 | N2a | T3 | 5.3 | 1 | 141 | 0 | |
| BR-45 | Placebo | Nx | T1b | 0.8 | 3 | 547 | 0 | |
| PG53V53 | GP2 | N0 | T2 | 4.6 | 1 | 79 | 0 | |
| AG10V10 | Placebo | N3c | T4 | 5.0 | 3 | 326 | | 0 |

These data suggest that a baseline immune response to GP2 through a positive response to a DTH skin test suggest that the patient is at higher risk of imminent recurrence. It is theorized that a positive baseline DTH skin test to GP2 may be evidence of an existing immune response to GP2 associated with residual disease, impending recurrence, or prior treatments.

### Example 3

Injection site reaction analysis in phase IIb trial of GP2

Methods: Patients were randomized and received GLSI-100 (500 mcg GP2+125 mcg GM-CSF) or control (GM-CSF) via 6 intradermal injections every 3-4 weeks for the first 6 months and 4 booster injections every 6 months. The magnitude of injection site reactions, which occurred in almost all patients, were assessed for the 10 doses administered by measuring the largest perpendicular diameters and the resulting orthogonal mean. DTH reactions were assessed in a similar manner at baseline and 6 months after treatment and have been shown to increase over time.

Results: DTH reactions at baseline and 6 months were consistently correlated with injection site reactions suggesting that injection site reactions may be interchangeable with immune response data (baseline p=0.6, p<0.001; 6mo p=0.4, p=0.009). After a single dose, the median orthogonal mean induration was 7.8 mm for GM-CSF patients and 34.0 mm for GLSI-100 patients (p=0.0036). Injection site reactions increased to 78 mm after the 4^{th} injection but remained over 50 mm for the duration of the injection series for those treated with GLSI-100. The magnitude of injection site reactions for patients treated with GLSI-100 was similar across HER2 status. However, HER2 3+ control patients had significantly smaller reactions to GM-CSF than HER2 1-2+ control patients with an average of 43.1 mm difference over all 10 doses.

Conclusions: This study demonstrated that GLSI-100 safely elicited a potent immune response, as evidenced by injection site reactions that correlate to and serve as a complement to immune response data such as DTH.

### Example 4

GP2 immune response is a predictor of recurrence in a phase IIb study

The study enrolled 180 patients with both HER2 3+ positive and low HER2 expressors (1-2+). After 5 years of follow-up, the Kaplan-Meier estimated 5-year DFS rate in the 46 HER2 3+(positive) patients treated with GLSI-100, if the patients were treated, followed, and remained disease free over the first six months of treatment, was 100% versus 89.4% (95% CI:76.2, 95.5%) in the 50 placebo patients treated with GM-CSF (p=0.0338).

A DTH reaction was used to assess in vivo immune responses in patients (n=150) prior to exposure to study medication and after 6 months of the first dose. The DTH orthogonal mean was measured 48-72 hours after injection using the sensitive ballpoint-pen method. GP2 DTH response increases in patients after treatment with GLSI-100.

Positive DTH immune responses to GP2 at baseline was defined as orthogonal mean induration larger than 5 mm. It was previously reported that 22.8% (33/145) of patients reacted to GP2 at baseline and that in the subgroup of subjects with recurrence, 36.4% (8/22) had a positive baseline DTH (Example 1).

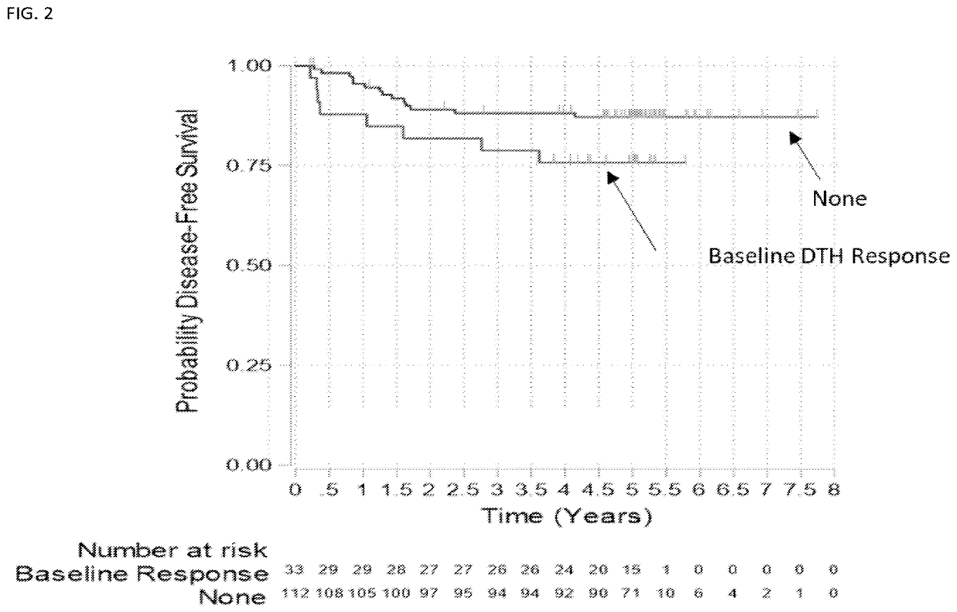

Further analysis shows that a baseline DTH response may be predictive of disease-free survival. The Kaplan-Meier log-rank test depicted in FIG. 2, in a pooled population of HER2 positive and HER2 low patients, showed a borderline effect (p=0.0956) of baseline DTH response on disease-free survival. At year 4, 88% of all patients without a positive baseline GP2 DTH remained disease-free. The patients with a positive baseline GP2 DTH recurred 3.5 years earlier reaching a similar survival probability of 88% at 6 months.

In addition, the proportion of subjects recurring at any time was 24.2% (8/33) amongst those with a positive baseline GP2 DTH response versus 12.5% (14/112) amongst those with a negative baseline GP2 DTH response (p=0.0984), suggesting that a positive baseline GP2 DTH may be associated with recurrence.

Conclusion: The probability of recurrence is increased and recurrence is likely to occur years sooner in subjects with a positive GP2 DTH immune response at baseline versus those subjects without a positive GP2 DTH immune response at baseline. Further studies assessing the prognostic value of GP2 immune response at baseline are planned. These studies may include additional measures of GP2 immune response by DTH.

### Example 5

Injection site reactions correlate to delayed type hypersensitivity tests

Injection site reactions (ISR) were measured 48-72 hours after injection. Delayed-type hypersensitivity (DTH) to GP2 was performed at baseline and after 6 months of treatment. For the DTH test, 0.5 mL consisting of 100 mcg of GP2 reconstituted in bacteriostatic saline for injection was placed intradermally. The DTH reaction was measured 48-72 hours after injection.

This trial explored HER2 3+(positive) patients, who generally received a standard course of trastuzumab after surgery, and HER2 1-2+ patients, who did not receive trastuzumab after surgery. The orthogonal mean of the largest diameter of the injection site was measured 48-72 hours after injection. A DTH reaction was used to assess in vivo immune responses in patients prior to exposure to study medication and after 6 months of the first dose. The DTH orthogonal mean was measured 48-72 hours after DTH placement using the sensitive ballpoint-pen method. DTH reactions increase after the PIS compared to baseline DTH reactions.

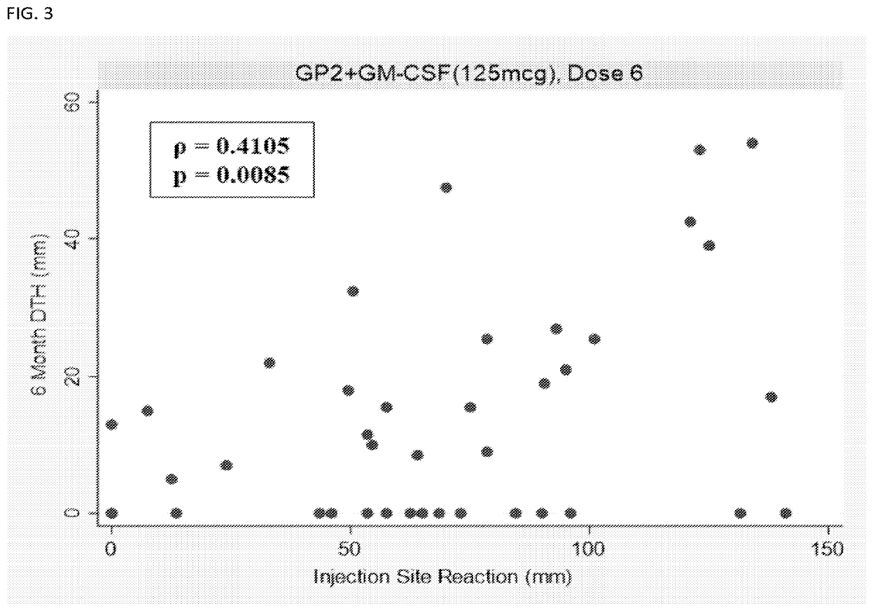

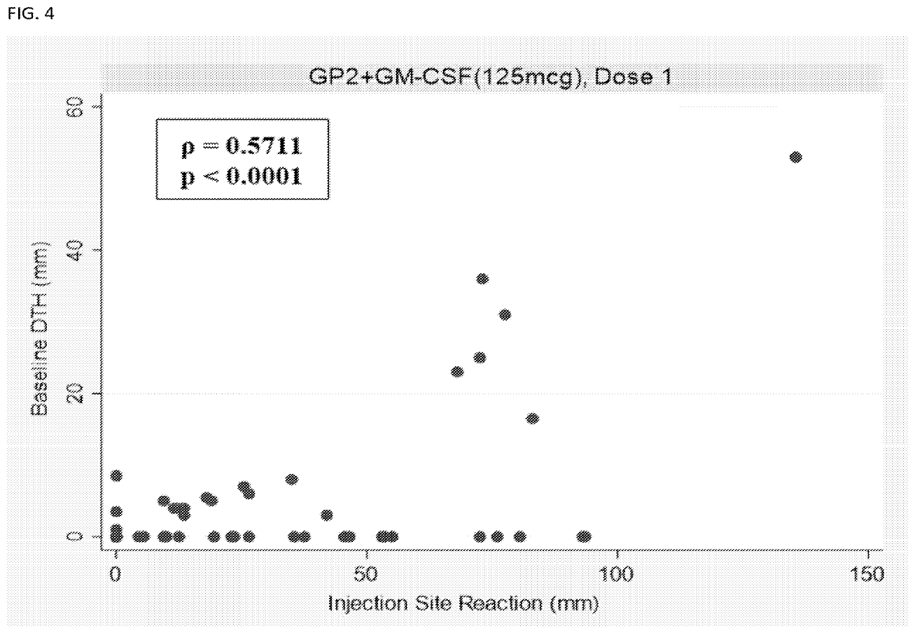

As shown in FIG. 3, in GLSI-100 treated subjects the DTH response at month 6 was found to be correlated with the ISR at the nearest dose, Dose 6 (P=0.41, p=0.0085). In addition, DTH response at month 6 was found to be correlated with most ISRs. As shown in FIG. 4, the DTH response at baseline correlated with the ISR at the nearest dose, Dose 1 (=0.57, p >0.0001).

Conclusions: This study demonstrated that GLSI-100 safely elicited a potent immune response, as evidenced by ISRs that correlate to and may serve as a complement to immune response data such as DTH.

Conclusions: This study demonstrated that injection site reactions correlate to DTH test reactions. Injection site reactions may complement or replace DTH tests or other immune response methods.

### Example 6

Baseline GP2 immune response as an independent prognostic factor in a phase IIb study

As shown in the examples above, subjects with baseline response to GP2 by DTH were more likely to experience a recurrence sooner than those without a positive baseline response. It was of interest to determine if this response was independent of other known prognostic factors.

GP2 DTH skin tests were placed at baseline and after the 6th dose. After 48-72 hours, the largest perpendicular diameters of induration were measured and the orthogonal mean was calculated. Induration of over 5 mm was considered a positive response. The relationship between prognostic factors such as demographic and disease characteristics and the baseline DTH response were investigated by logistic regression.

Known prognostic factors such as hormone receptor status, stage at presentation, node status, etc. were evenly distributed across subjects that experienced a baseline DTH response to GP2 and those who did not. Each prognostic factor was investigated in a logistic model to determine if it predicted baseline DTH immune status. The prognostic factors did not predict baseline DTH immune response in the logistic models. There appears to be no correlation between baseline immune response to GP2 and known prognostic factors.

Conclusions: Baseline GP2 immune response as measured by the delayed-type hypersensitivity test may be an independent prognostic factor for recurrence. Knowledge of this GP2 immune response may identify a patient with increased risk of rapid recurrence.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The disclosure is not limited to the exact details shown and described, for variations obvious to one skilled in the art will be included within the disclosure defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present disclosure. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A method comprising:
testing a subject's immune response to a peptide of a protein,
wherein the subject had a cancer,
determining that the subject has a positive immune response to a peptide of an oncogenic protein correlating with the cancer,
wherein the testing occurs at baseline, after baseline, or before baseline,
wherein the positive immune response indicates the subject is at increased risk of recurrence of a cancer, a cancer is present in the subject, the subject had a cancer prior to the testing and developed an immune response to the peptide, or a combination thereof, or the subject has developed an immune response to the peptide for any other reason.

2. A method for determining time to recurrence of a cancer, comprising:
testing a subject's immune response to a peptide of a protein correlating with a cancer,
wherein the subject had the cancer,
determining that the subject has a positive immune response to the peptide,
wherein the presence of an immune response to the peptide is indicative of a shorter time to recurrence of the cancer than the absence of the response.

3. A method for identifying the presence of a cancer, comprising:
testing a subject's immune response to a peptide of a protein correlating with a cancer,
determining that the subject has a positive immune response to the peptide related to a current cancer that the immune system is responding to and thus generating an immune response to the peptide.

4. The method of claim 1, wherein the peptide is at least 6 amino acids.

5. The method of claim 1, wherein the peptide is a peptide of an oncogenic protein.

6. The method of claim 5, wherein the oncogenic protein is HER2/neu.

7. The method of claim 6, wherein the peptide is GP2.

8. The method of claim 5, wherein the cancer comprises HER2/neu expressing cells.

9. The method of claim 8, wherein the HER2/neu expressing cells are HER2/neu 3+, HER2/neu positive, HER2/neu intermediate, or HER2/neu low.

10. The method of claim 1, wherein the subject had or has breast cancer.

11. The method of claim 1, wherein the positive immune response indicates a cancer is present in the subject or the cancer for which the subject is at increased risk of recurrence is breast, and wherein the cancer is metastatic cancer.

12. The method of claim 1, wherein the testing comprises a delayed type hypersensitivity (DTH) immune response to the peptide.

13. The method of claim 12, wherein the peptide is a peptide of an oncogenic protein.

14. The method of claim 13, wherein the oncogenic protein is HER2/neu.

15. The method of claim 14, wherein the peptide of the oncogenic protein is GP2.

16. The method of claim 14, wherein the subject had or has breast cancer, ovarian cancer, gastric cancer, cervical cancer, or any other HER2/neu expressing cancer.

17. The method of claim 14, wherein a positive DTH immune response is indicative of HER2/neu-expressing cancer cells in the subject, increased risk of recurrence of a HER2/neu-expressing cancer in the subject, the subject had a HER2/neu-expressing cancer prior to the testing, or a combination thereof.

18. The method of claim 1, wherein the subject is undergoing treatment with a therapeutic peptide against a cancer, and wherein the testing comprises evaluation or measurement of an injection site reaction.

19. The method of claim 18, wherein the testing comprises measuring the largest perpendicular diameters and the resulting orthogonal mean.

20. The method of claim 19, wherein the peptide is a peptide of an oncogenic protein.

21-34. (canceled)


SEQUENCE LISTING (1 sequence)

SEQ ID NO: 1 - protein, 9 aa, synthetic construct
IISAVVGIL